SEQUENCE LISTING (1 sequence)

SEQ ID NO: 1 - protein, 1235 aa, synthetic construct
  source 1..1235: note = Severe acute respiratory syndrome virus type 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGYIPEAPRDGQAYVRKDGEWVLLSTFLLEVLFQGP

(19) United States
(12) Patent Application Publication
(10) Pub. No.: US 2023/0210982 A1
(43) Pub. Date: Jul. 6, 2023

(54) AUTOLOGOUS DENDRITIC CELL VACCINE KIT AND USES

(71) Applicant: AIVITA BIOMEDICAL, INC, Irvine, CA (US)

(72) Inventors: Gabriel NISTOR, Irvine, CA (US); Hans KIERSTEAD, Laguna Beach, CA (US); Robert O. DILLMAN, Irvine, CA (US)

(21) Appl. No.: 18/061,045

(22) Filed: Dec. 2, 2022

Related U.S. Application Data
(63) Continuation of application No. PCT/US21/35501, filed on Jun. 2, 2021.
(60) Provisional application No. 63/033,678, filed on Jun. 2, 2020.

(51) Int. Cl. A61K 39/215; C12N 5/0784

(52) U.S. Cl. CPC A61K 39/215; C12N 5/0639; C12N 2501/2304; C12N 2501/22; C12N 2501/2302; C12N 2501/24; A61K 2039/5154

(57) ABSTRACT

Disclosed herein is a kit to produce a personalized vaccine based on autologous dendritic cells. The kit contains all the materials, reagents and information necessary to produce a dose of live dendritic cell vaccine against a pathogen organism, part of a pathogen organism, a toxin, a venom, a structure obtained by recombinant method or chemical synthesis.

### CROSS-REFERENCE TO RELATED APPLICATIONS

This application is a continuation of International Application No. PCT/US2021/035501 entitled “AUTOLOGOUS DENDRITIC CELL VACCINE KIT AND USES”, filed Jun. 2, 2021, which claims the benefit of U.S. Provisional Application No. 63/033,678, entitled “AUTOLOGOUS DENDRITIC CELL VACCINE KIT AND USES”, filed on Jun. 2, 2020, each of which is incorporated by reference herein in its entirety.

### SEQUENCE LISTING

The instant application contains a Sequence Listing which has been submitted electronically in XML format and is hereby incorporated by reference in its entirety. Said XML copy, created on Nov. 18, 2022, is named 56805-711_301_SL.txt and is 3,348 bytes bytes in size.

### BACKGROUND

Personalized vaccines have received great research interest, but there has been limited translation into clinical practice. Production of personalized vaccines has typically required the equipment and trained personal of a research institution or dedicated pharmaceutical manufacturing facility, thereby limiting their availability, contributing to high cost, and impeding their adoption. Personalized vaccines have been pursued primarily for cancer treatment, where their high cost is more readily borne.

### SUMMARY

A simple vaccine kit that has all the components necessary to produce a dendritic cell-based autologous vaccine allows rapid production of vaccines, including in emergency situations. The dendritic cell-based vaccine prepared ex vivo avoids the possibility of toxicity of antigens as well as possible immune tolerance induction. The herein disclosed personal autologous vaccine kit enables vaccine production in facilities such as community hospitals having only basic laboratory equipment.

Disclosed here is a composition of an ensemble of components, or a kit, that are necessary for every step of making an autologous vaccine.

Also disclosed herein are methods of use of a vaccine manufacturing kit to produce a personal vaccine against an antigen.

One aspect is a kit for making a personalized dendritic cell (DC) vaccine for an individual. The kit comprises a kit container to contain the other components of the kit. The components of the kit include blood collection supplies, monocyte separation media or an inertial separation device, DC differentiation media components, a cell culture container, indicia of unique identity, and an antigen.

In some embodiments, the monocyte separation media is contained in a blood collection vacuum tube. In some embodiments, the monocyte separation media is FICOLL®, a neutral, highly branched, high-mass, hydrophilic polysaccharide.

The DC differentiation media components comprise a basal cell culture medium, such a RPMI-1640, PRIME-XV Dendritic Cell Maturation chemically-defined medium, or AIM-V media. In some embodiments, a bicarbonate-free, CO₂-independent version of the media is used. Is some embodiments, the HEPES is used as a CO₂-independent buffer. In some embodiments, the media contains interleukin 4 (IL-4). In some embodiments, the media contains IL-4 and granulocyte-macrophage colony-stimulating factor (GM-CSF). In some embodiments, the media does not contain GM-CSF. In some embodiments, the media contains interferon alpha (IFNα), In some embodiments, the media contains interferon gamma (IFNγ). In some embodiments, the media contains interleukin 2 (IL-2). In some embodiments, the media contains up to 30% autologous plasma.

In some embodiments, the cell culture container is a closed system with at least one access port. In some embodiments, the cell culture container is a bag, while in other embodiments it is a rigid vessel with a flat inner surface. In some embodiments, the interior surfaces are hydrophobic.

In various embodiments, the indicia of unique identity can be a string of alphanumeric characters, a bar code, or a QR code.

In some embodiments, the antigen is a whole pathogen organism, or a fragment derived from a pathogen organism such as a bacteria, fungus, virus, rickettsia, mycoplasma, or parasite.

In some embodiments, the antigen is a toxin or a venom. Non-limiting examples include toxins and venoms derived from bacteria, insects, and plants, or synthetically made chemical compounds.

In some embodiments, the antigen is a purified molecule such as a protein or peptide, or a fragment thereof. In some embodiments, the antigen is produced by recombinant technology.

In some embodiments, the antigen is produced by chemical synthesis.

In some embodiments, the antigen is the full length spike protein of SARS-CoV-2.

In some embodiments, the kit container is capable of serving as an incubator. In some embodiments, the kit container has insulated walls. In some embodiments, the kit container has a rechargeable power supply, for example a lithium battery. In some embodiments, the power supply is a lithium polymer battery that can be shaped to fit the kit container. In some embodiments, the kit container comprises a thermostat, but not a temperature controller. In some embodiments, the thermostat comprises a phase exchange material and a positive temperature coefficient material.

One aspect is a method of making a personalized, autologous DC vaccine using a herein disclosed kit. In some embodiments, the method comprises collecting blood from an individual, isolating peripheral blood mononuclear cells (PBMC), differentiating the PBMC to generate immature DC by adding the DC to the cell culture container and incubating the cells for 2-5 days, then adding antigen to the cell culture container to load the immature DC with antigen and incubating for a further 1-2 days (wherein the antigen will serve as an immunogen to induce an immune response against a component of the pathogen, toxin or venom that is the target of the vaccine), and harvesting antigen-loaded immature DC.

Some embodiments of the methods of making the personalized, autologous DC vaccine further comprise reserving autologous plasma from the isolating step. Some embodiments of the methods of making the personalized, autologous DC vaccine further comprise affixing indicia of unique identity to a container containing cells or plasma from the individual. Some embodiments of the methods of making the personalized, autologous DC vaccine further comprise re-suspending the harvested antigen-loaded immature DC in autologous plasma.

Some embodiments of the methods of making the personalized, autologous DC vaccine further comprise storing the harvested, re-suspended antigen-loaded immature DC prior to administration to the individual. Some embodiments comprise storing the harvested, re-suspended antigen-loaded immature DC at room temperature for up to 5 hours. Some embodiments comprise storing the harvested, re-suspended antigen-loaded immature DC at 4° C. for up to 48 hours. Some embodiments comprise storing the harvested, re-suspended antigen-loaded immature DC at −80° C. for up to 21 days.

One aspect is a personalized, autologous dendritic cell (DC) vaccine made by any of the herein disclosed methods of making such a vaccine.

One aspect is a method of immunizing an individual comprising administering a herein disclosed personalized, autologous DC vaccine to the individual. In some embodiments, the personalized, autologous DC vaccine is administered by subcutaneous injection. In some embodiments, the personalized, autologous DC vaccine is administered by intradermal injection. In some embodiments, immunization is achieved with a single administration.

### BRIEF DESCRIPTION OF THE DRAWINGS

FIG. 1 depicts the principle mechanism of the autologous dendritic cell vaccine. A blood sample is obtained from a subject, monocytes are isolated from the blood sample and in vitro differentiated into dendritic cells that are loaded with antigens and re-injected into the same person from which the sample was obtained.

FIG. 2 depicts the reactivity of endogenously-produced IgG antibodies against recombinant viral spike protein in patients infected with SARS-CoV-2 virus.

FIG. 3 depicts the reactivity of endogenously-produced IgG antibodies against recombinant viral nucleocapsid protein in 11 different patients infected with SARS-CoV-2 virus.

FIG. 4 depicts the reactivity of endogenously-produced IgA antibodies against recombinant viral spike protein in 11 different patients infected with SARS-CoV-2 virus.

FIG. 5 depicts the reactivity of endogenously-produced IgA antibodies against recombinant viral nucleocapsid protein in 11 different patients infected with SARS-CoV-2 virus.

FIG. 6 depicts flow cytometric data demonstrating that monocytes differentiate in high proportion into CD11c⁺ and CD14 negative dendritic cells.

FIG. 7 depicts cytokine secretion in the autologous mixed lymphocyte coculture with antigen loaded dendritic cells versus control.

FIG. 8 depicts the percent increase of cytokine secretion in the autologous mixed lymphocyte coculture with antigen loaded dendritic cells at various antigen-loading amounts.

FIG. 9 depicts flow cytometric data demonstrating that Tbet transcription factor positive lymphocytes increase after exposure to antigen-loaded dendritic cells.

FIG. 10 depicts the steps of one general manufacturing process for an autologous DC vaccine disclosed herein.

FIGS. 11A-B depict the proportion of SARS-CoV-2 spike protein-specific IFNγ secretory lymphocytes in the PBMC population before (FIG. 11A) and 2 weeks after (FIG. 11B) immunization with an autologous DC vaccine specific for the spike protein of SARS-CoV-2 as determined by ELISpot assay.

FIG. 12 presents the average spot count for SARS-CoV-2 spike protein-specific IFNγ secretory lymphocytes in the PBMC population before and 2 weeks after immunization with an autologous DC vaccine specific for the spike protein of SARS-CoV-2 as determined by ELISpot assay with or without antigenic stimulation in the assay.

FIGS. 13A-B depict an assessment of memory response. FIG. 13A shows the percentage of subjects exhibiting a memory response to SARS-CoV-2 spike protein before and 2 weeks after immunization with an autologous DC vaccine specific for the spike protein of SARS-CoV-2 as determined by ELISpot assay for SARS-CoV-2 spike protein-specific IFNγ secretory lymphocytes, with antigenic stimulation in the assay. FIG. 13B shows the number of subjects according to memory status, converted, boosted, or no change, at 2 weeks after immunization.

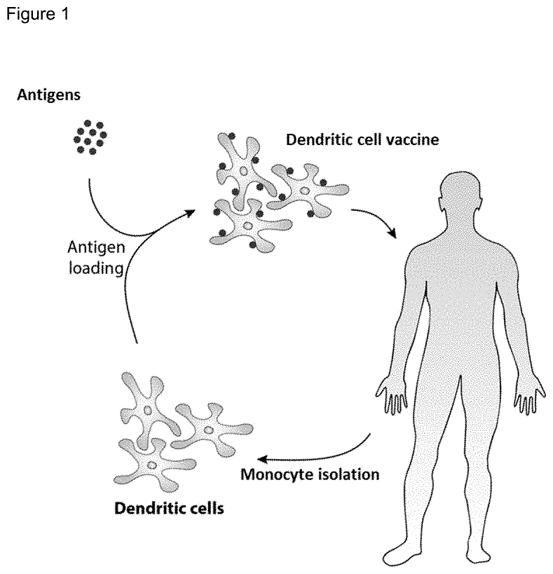

### DETAILED DESCRIPTION

Although demonstrated safe, efficacious, and superior to other vaccine approaches in research settings, the personalized dendritic cell (DC) immunotherapy presents the unsolved challenge of industrial manufacturing scale-up. The compositions and methods disclosed herein address the scale-up challenges and provide a feasible solution for industry.

DC processing and presentation of viral antigens is well-established with immune effects demonstrated in vitro and in animal models. Vaccines using dendritic cells have demonstrated protection against leishmaniasis, Herpes Simplex virus, influenza virus, Candida albicans, and human immunodeficiency virus (HIV). In a model of APC-specific immunosuppression, DC-based influenza vaccines rapidly induced significant antigen-specific antibody titers which protein vaccination could not achieve.

Application of DC immunotherapy as prophylaxis for infectious diseases is both scientifically rational and cost effective, especially for certain niche populations who are at risk of increased morbidity and who respond poorly to traditional vaccination.

DCs naturally phagocytose and digest soluble antigens for presentation to other immune cells. In this process, particles are endocytosed after cell surface receptor recognition or by micropinocytosis or by non-selective endocytosis of solutes. The uptake of antigens results in activation signals that lead to DC maturation to facilitate antigen presentation and maximal stimulation of cells for the adaptive immune response. Human monocyte-derived DCs and monocyte-derived macrophages can be generated from monocytes in vitro. Culturing monocytes with GM-CSF and IL-4 gives rise to DCs, while culturing with either macrophage colony-stimulating factor (M-CSF) or GM-CSF alone creates macrophages. Monocyte-derived DCs are excellent antigen presenters and induce antigen-specific CD4⁺ and CD8⁺ T cells. They express multiple pattern recognition receptors (PRRs), such as toll-like receptors (TLR) and C-type lectin receptors (CLRs), to recognize pathogen-associated molecular patterns (PAMPs), damaged-associated molecular patterns (DAMPs) or altered glycosylated self-antigens such as tumor antigens. TLR recognition induces intracellular signaling and expression of antigen-presenting molecules (MHC II molecules), co-stimulatory molecules (CD80/86, CD40), inflammatory and/or antiviral cytokines (such as TNF-α, IL-12, IL-23, IFNα/β) and chemokines (i.e., IL-8, RANTES).

Vaccines using non-viable or attenuated pathogens require large amounts of antigen and extensive testing. A DNA/RNA vaccine is a reasonable alternative because the antigen would undergo immediate MHC presentation; however, an effective delivery system remains a major challenge. The genomic delivery system could be toxic, immunogenic and prevent any future use of the same carrier (for example, an adenovirus or adeno-associated virus vector).

Pattern recognition receptor expression varies with the differentiation and maturation of mononuclear phagocytes. We have established culture conditions that produce immature DCs with a high capacity for antigen uptake and cross-presentation. Ex vivo maturation is facilitated by exposure to TLR4 ligands (LPS, Poly I:C). After peripheral injection (subcutaneous or intradermal), further maturation is accomplished in vivo during migration to regional lymph nodes.

Autologous immature DCs loaded ex vivo to facilitate uptake, processing, and presentation of viral antigen can overcome inhibition caused by some pathogens, such non-limited examples include coronaviruses, HIV, influenza, Ebola, HSV-1, measles, hepatitis C, Dengue virus, and others. Avoiding inhibitory pathways, ex vivo antigen processing enhances induction of humoral and cellular immune responses against viral antigens.

The herein presented autologous vaccine containing immature DCs loaded ex vivo with antigen provides the advantage of Th1-type immune biasing mediated by effector CD8⁺ cytotoxic cells, an essential mechanism that is superior to Th2 response in countering viral infections.

A cell-mediated response by effector CD8⁺ lymphocytes does not have to be targeted to a particular antigenic epitope; a response to any immunogenic epitope in any expressed antigen can be of benefit. In comparison, antibody-mediated immunity must be directed against specific epitopes of a viral surface antigen. In many cases, neutralizing antibodies are directed against the receptor binding site of the viral receptor, though neutralization can involve blocking uncoating or, for envelope viruses, lysis. Accumulation of non-neutralizing antibody causes an increased risk of antibody enhanced disease when the receptor area mutates and the neutralizing antibodies are ineffective such as in the most recent corona virus (SARS-CoV-2) pandemic.

Regardless of the epitope recognized, cell mediated immunity is always neutralizing and will target infected host cells, thereby impeding further viral replication.

The disclosed DC vaccine product is much more accommodating of mutations than would be anti-receptor binding domain antibodies that are the goal of a classic vaccine optimized by its adjuvant system to maximize humoral response.

Certain viruses, such as SARS-CoV-2 are known to inhibit cell mediated immunity with severe reduction of circulating CD8⁺ cells and suppression of interferon production. The immune response generated by these viruses is mostly humoral, first by production of antibodies against non-mutated epitopes and 7-14 days later against the mutated parts of the virus. If the mutations are in the receptor binding domain (RBD), there is a 7-14 day window of viral propagation and non-neutralizing antibody production. This phenomenon is causing innate immune cell activation and a general inflammation particularly where the receptors for the virus are more abundant and that explains the acute respiratory distress syndrome (ARDS) pathology that occurs in some patients with other comorbidities conducive to a Th2 bias (age, diabetes, obesity, etc.)

### Manufacturing Process

The manufacturing process disclosed herein is based on individual single use kits assigned to a single individual. The kit contains all the material and the reagents need for manufacturing and quality control of the finished product.

The kit typically includes: blood collection supplies, monocyte separation media, DC differentiation media, plasticware, dose containers, QC sampling containers, QC reagents labels, and documentation. Nonetheless, other arrangements are possible.

Each kit component is uniquely identified, recorded in manufacturing documentation and traceable according to current regulatory provisions. In various embodiments, the unique indicia of unique identity of individual kits can be any of a string of alphanumeric characters, a bar code, a QR code, and the like. In some embodiments, the indicia are printed on stickers that can be affixed to components of the kit as they are utilized and in hard-copy patient records.

In some embodiments, the kit container, in addition to containing the various components during storage and shipping, can also serve as an incubator.

The blood collection is accomplished by using standard heparinized vacuum tubes and a phlebotomy kit. The collection tube size varies between 5 mL and 50 mL and optionally includes a Ficoll separation layer in each tube. One method of collection is using vacuum tubes preloaded with separation media.

An alternative method of collection uses an inertial microfluidic device for blood separation. Such devices use a non-equilibrium inertial separation array.

The peripheral blood mononuclear cells (PBMC) are concentrated by centrifugation or tangential flow filtration in a same closed loop system. A small amount of plasma is collected and stored separately (reserved) for the final product composition. In some embodiments, heparin is used as the anticoagulant.

The isolated PBMCs are then exposed to a dendritic cell differentiation media for 2-5 days.

An exemplary media for DC differentiation is a CO₂ independent formulation containing a non-bicarbonate buffer. Examples of a CO₂ independent media formulation is RPMI-1640, bicarbonate free, and AIM V medium (ThermoFisher). One example of a non-bicarbonate buffer suitable for tissue culture is HEPES.

In some embodiments, the DC differentiation media contains antioxidants and free radical scavengers. Examples of free radical scavengers include N-acetyl-cysteine, carboxy-PTIO, flavonoids, and L-NG-methylarginine.

In some embodiments, the DC differentiation media contains GM-CSF and IL-4.

In some embodiments, the DC differentiation media does not contain GM-CSF.

In some embodiments, the DC differentiation media contains IFNγ.

In some embodiments, the DC differentiation media contains IFNα.

In some embodiments, the DC differentiation media contains IL-2.

In some embodiments, the DC differentiation media may contain up to 30% autologous plasma that was reserved during blood collection and PBMC isolation process. In some embodiments, the media contains 5% to 30% autologous plasma, or any integer value in that range, inclusive.

The PBMC cell suspension is then transferred in a closed system cell culture container for DC differentiation and antigen exposure.

An exemplary closed system cell culture container is a flexible bag with an inner cell culture surface of approximately 50 cm² and at least one access port.

An alternative closed system cell culture container is a rigid container with a flat inner cell culture surface of approximately 50 cm² and an access port.

The closed system cell culture container inner surfaces are hydrophobic to prevent cell attachment

An exemplary material for the closed system cell culture container is a gas permeable material, such as fluorinated poly-ethylene and its copolymers.

An alternative for the closed system cell culture container material is cellulose that is chemically modified for hydrophobicity. In various embodiments, the chemically modified cellulose is cellulose acetate that is acylated or esterified with a fatty acid (e.g., palmitate, stearate, etc.).

Alternatively, the hydrophobic property can be achieved by coating the inner surface with a hydrophobic material. Such material can be for example a hydrophobic silane.

The container containing the PBMCs and media are incubated at 37° C. for 2-5 days.

An exemplary incubator is a kit container that contains a rechargeable power supply and a thermostat. The kit container walls provide thermal insulation that allows minimal energy dissipation to maintain the content at about 37° C. for 5-7 days. In some embodiments, the power supply consists of a lithium polymer battery that can be shaped to fit the box.

An exemplary thermostat includes a phase exchange material and/or a positive temperature coefficient (PTC) material that eliminates the need for a temperature controller. As used herein, a thermostat is any system that maintains a constant temperature (within a tolerance) while a temperature controller is a mechanical or electronic system that has a sensor and a feedback loop to enable or disable infusion of energy (heat). As used herein, a phase exchange material is a substance which releases or absorbs enough energy during phase transition to provide heating or cooling. For example, as heating raises the temperature of a solid phase exchange material to the melting temperature, heat is absorbed with nearly no change in temperature until all of the material is melted. Similarly, as heat is dissipated and the temperature of a liquid phase falls toward the melting point, the material solidifies and heat is released with nearly no change in temperature until all of the material is solidified. In some embodiments, the phase exchange material is Paraffin 20-Carbons (melting point 36.7° C.). In some embodiments, the phase exchange material is camphenilone (melting point 39° C.). In some embodiments, the phase exchange material is referred to as a means for releasing or absorbing energy or means for latent heat storage.

A PTC material exhibits increased electrical resistance as temperature rises. A PTC material can be designed to reach a maximum temperature for a given input voltage, since at some point any further increase in temperature would be met with greater electrical resistance. Unlike linear resistance heating or negative temperature materials, PTC materials are inherently self-in some embodiments, the PTC material is a silicone rubber which conducts electricity with a resistivity that increases exponentially with increasing temperature for all temperatures up to a temperature where the resistivity grows to infinity. Above this temperature the PTC rubber is an electrical insulator. In particular, PTC rubber can be made from polydimethylsiloxane (PDMS) loaded with carbon nanoparticles. In some embodiments, The PTC material is a carbon-based PTC ink. The PTC ink is deposited on the exterior surface of the cell culture container. In some embodiments, PTC materials are referred to as means for PTC limited heating.

In various embodiments, the thermostat comprises a phase exchange material, or a PTC material, or both. In some embodiments, the thermostat is referred to as means for maintaining constant temperature. In some embodiments, the constant temperature is about 37° C.

The immature dendritic cells are then exposed to the antigen for 1 or 2 days. Antigen can be added to the closed system cell culture container through a compliant port, for example, a self-sealing swabbable valve. In some embodiments, the media is never changed throughout the 3-7 day culture period.

Suitable antigens for DC loading include soluble or insoluble antigens derived from pathogens, toxins, and venoms or insoluble antigens that include whole, live-attenuated, nonviable, fragments of pathogens, or protein complexes. A variety of synthetic or recombinant structures engineered from DNA/RNA sequences of the pathogens can be used as antigen sources. Furthermore, the antigens can be fused with terminal peptide sequences that enhance the antigenicity or stimulate the dendritic cells. The fused peptide sequences can include for example fragments of human immunoglobulins and chemical structures that activate toll-like receptors (TLR) of the dendritic cells.

As published elsewhere, a recombinant method can be used to produce antigens. The recombinant methods use sequences derived from the DNA/RNA analysis and chemical structure of the pathogen. The anticipation of antigenicity can be also based on human HLA matching on major and minor subclasses.

The validation of the antigen targets can be performed by an antibody binding test from a survivor of infection with the pathogen or a convalescent patient that was confirmed positive for the targeted pathogen.

A combination of antigens can be used to produce a vaccine with a broader protection.

For dose preparation, the cell content of the container is collected in a centrifuge tube and the supernatant is removed by centrifugation and replaced with autologous plasma.

If freezing of the dose before administration is anticipated, a cryopreservative solution can be added at this step. In some embodiments, the cryopreservative is mixed with an equal volume of autologous plasma to re-suspend the cells. An exemplary cryopreservative contains trehalose and glycerin. Use of trehalose and glycerin as cryopreservatives allows for direct injection of the thawed product, as they are USP listed for vaccine adjuvants. Some other cyropreservatives, such as DMSO, would have to be removed before the thawed product could be injected.

A small amount of cells are optionally subjected for quality control. In some embodiments, the cell population contains 5-30% dendritic cells and 70-95% lymphocytes, and the content of non-differentiated monocytes is less than 1%. The absence of macrophages indicates the effectiveness of the DC differentiation, and that removal of adherent cells prior to DC differentiation is not necessary.

In some embodiments, the dose does not contain residual antigen or cell culture media.

The dose is then transferred into final container that is stored at room temperature for immediate use, at 4-8° C. for use in the following 2 days, at −65 to −85° C. for up to 21 days and in liquid nitrogen (<−165° C.) for long term storage.

The quality control may include an identity test, for example testing for CD14-CD11+ cells; a safety testing for microbiological contamination (mycoplasma, endotoxin and sterility) and a potency assay, for example presence of IL12 in the supernatant.

An exemplary potency assay includes a rapid evaluation method such as a lateral flow immunoassay.

An exemplary rapid sterility testing is based on solid-phase laser scanning cytometry to rapidly enumerate viable microorganisms from aqueous samples. The method is included in commercially available devices such as ScanRDI® (bioMérieux Inc).

In some embodiments, the dose is verified with the patient identity and administered by s.c. injection.

### Process and Product Distribution

The production and distribution of the vaccine product may be coordinated from a central location. In some embodiments, the personalized DC vaccines are produced in low technology laboratories that use common biotechnology or clinical laboratory settings and skills. In some embodiments, the production and distribution of the vaccine product is conducted at the same location.

In a first step, the laboratory is evaluated for space, equipment, and personnel qualification. The typical laboratory would include a biosafety level 2 space that include an incubator, centrifuge, refrigerator, freezer, microscope, and common laboratory instruments.

In some embodiments, a specialized software module is implemented in the facilities' existing quality management system (QMS). The software provides instructions, manufacturing records, control, and release procedures in compliance with the United States Food and Drug Administration or equivalent regulatory institutions in other countries. This module communicates with the centralized QMS at the coordinating center to ensure availability of manufacturing kits, training and troubleshooting.

A production capacity for each location is evaluated and manufacturing kits are planned or reserved accordingly. The supply chain for each component is contractually secured.

The kits may be manufactured according to a bill of materials by a specialized third party assembling and warehousing organization and distributed according to local production.

An exemplary kit format includes all reagents as dry substances already included in the cell culture container, the reconstitution of the liquid format is performed by adding the cells suspended in sodium chloride 0.8% solution after PBMC isolation. For embodiments in which the DC differentiation media contains autologous plasma, the autologous plasma is also added to the cell culture at this point.

Once the antigen-loaded immature DC are resuspended they are ready to use, but it is expected that some period of storage will be required; for example, quality control assays can typically be completed in about 3 hours. Additionally, the patient will not necessarily be present at the facility where the vaccine is prepared. The vaccine can be stored at room temperature for at least up to 5 hours, at 4° C. for at least up to 48 hours, or at −80° C. for at least up to 21 days to allow for travel time of the patient and/or shipment of the vaccine.

### EXAMPLES

### Example 1. Generation of Dendritic Cells from an Individual Blood Sample and Loading with Recombinant Antigens Derived from SARS-CoV-2 Viral Genome

In a first step, the targeted antigens were validated for reactivity with naturally produced antibodies in known COVID-19 patients. For this purpose, serum from consented donors previously diagnosed with SARS-CoV-2 infection with a PCR tests was collected and exposed to ELISA plates that were coated with recombinant peptides obtained from the DNA sequence of the spike protein and nucleocapsid proteins of SARS-CoV-2 virus.

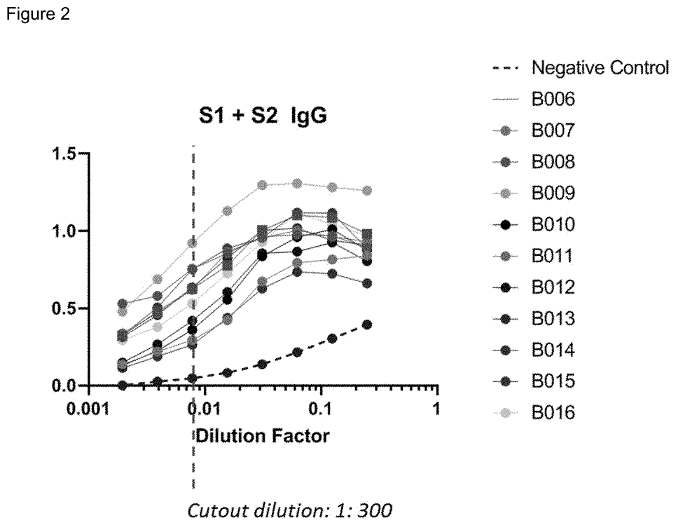

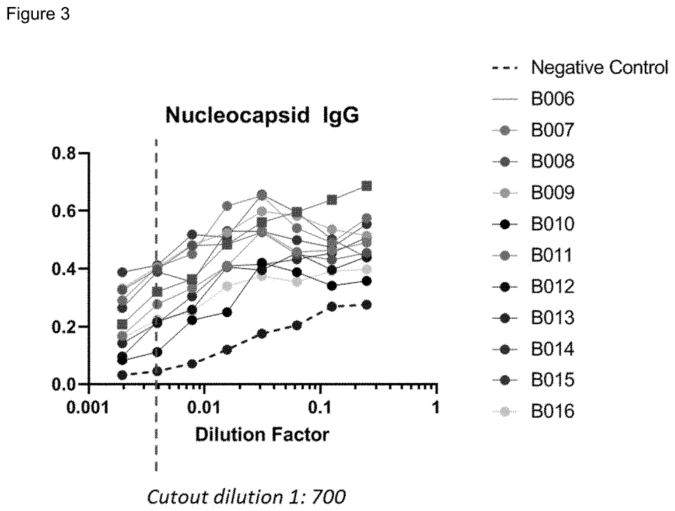

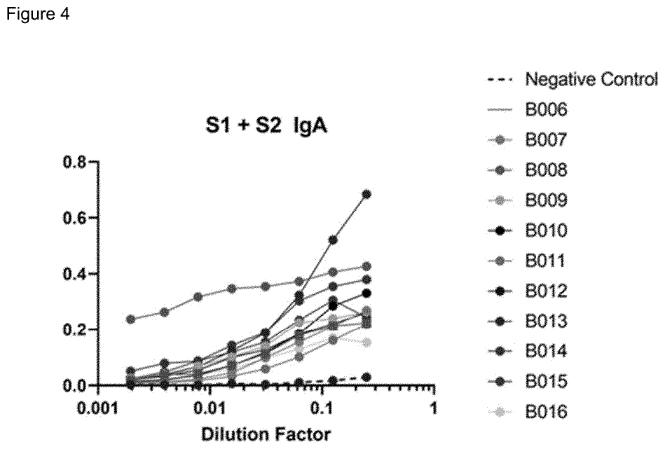

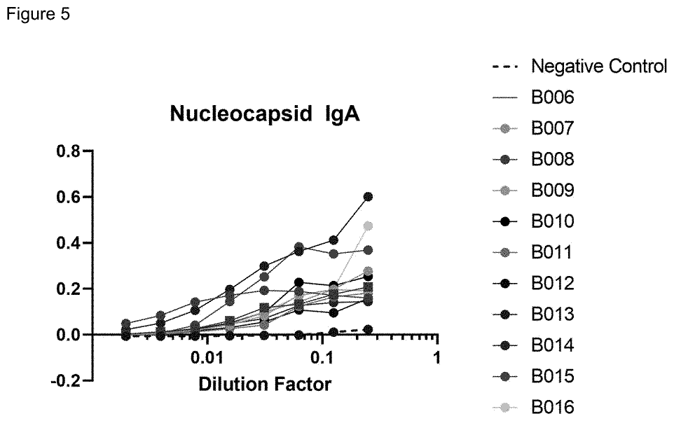

The analysis of the ELISA plates depicted in FIG. 2-5, confirmed the reactivity with the recombinant antigens.

Following antigen validation, four voluntary donors were subjected to a collection of 50 mL whole blood in heparinized vacutainers in order to produce an autologous DC vaccine.

A complete blood count (CBC) was performed to assess the starting mononucleate cell population. All volunteers had CBC values in normal range.

Within 6 hours of collection the whole blood underwent Ficoll separation. Part of the mononucleate cells were transferred in a 75 cm² cell culture dish, part of the cells were distributed in 12-well plates.

The non-adherent cells, consisting mainly of lymphocytes, were removed and preserved for later use.

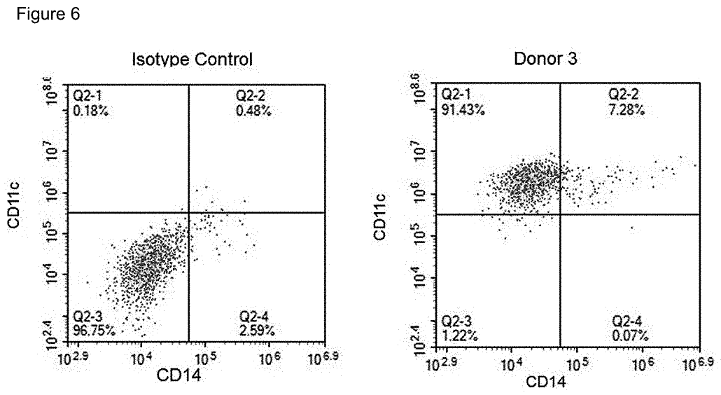

The adherent population was incubated with AIM-V media containing GM-CSF and IL-4 for 5 days. The monocytes differentiated in dendritic cells in a proportion of 90% population of CD11c positive/CD14-cells (FIG. 6).

The media was then removed and replaced with fresh same media that contained SARS CoV-2 antigens, derived by a recombinant method originated from the DNA sequence of spike proteins (S1, S2) and nucleocapsid, 3 μg from each per patient culture.

After 2 days, the loaded dendritic cells were sampled for phenotype, and re-mixed at a 1:3 proportion with the corresponding autologous lymphocytes.

The DCs and autologous lymphocytes were co-cultured for 72 hours and analyzed for markers that suggest lymphocyte activation.

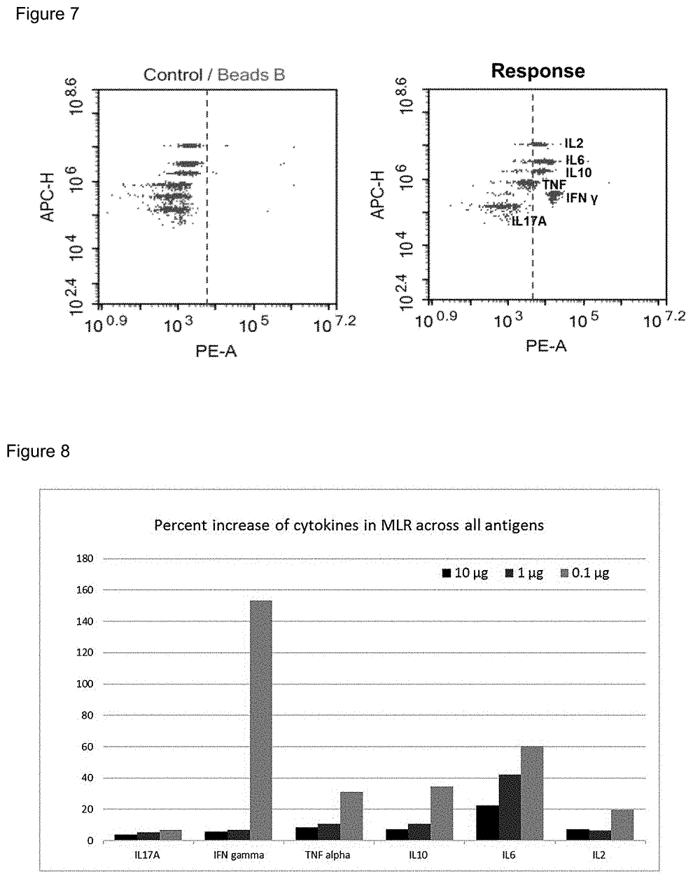

The results show the successful differentiation of monocytes in dendritic cells, the lack of toxicity of the antigens, and the cytotoxic activation of the lymphocytes co-cultured with the loaded dendritic cells (Table 1, FIG. 7 and FIG. 8).

TABLE 1
Cytokine secretion in the autologous mixed lymphocyte
coculture with antigen loaded dendritic cells at various
antigen concentration, averaged from 4 patients

| Antigen | | | IFN | TNF | | | |
|---|---|---|---|---|---|---|---|
| Dose | | IL17A | gamma | alpha | IL10 | IL6 | IL2 |
| 10 | μg | 3.82 | 5.69 | 8.43 | 7.46 | 22.37 | 7.10 |
| 1 | μg | 5.23 | 6.76 | 10.88 | 10.53 | 42.18 | 6.64 |
| 0.1 | μg | 7.02 | 153.22 | 31.12 | 34.65 | 60.42 | 19.68 |

As compared to control, where the differentiated DCs were not exposed to antigens, the CD8⁺ population increased to 41% after exposure to antigen.

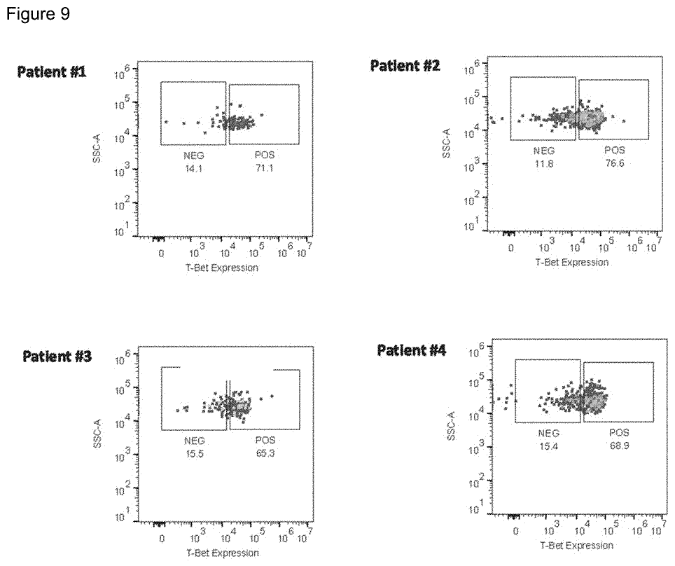

The CD4 helper population displayed a significant activation observed as Tbet transcription factor positivity averaging 17% increase over no-antigen control (FIG. 9) and lack of immune-toleration by absence (average 0.06%) of FoxP3 positive cells. Tbet positive cells are responsible for both Th1 and Th2 activation of the adaptive immune system.

These experiments demonstrate that the recombinant antigens are valid targets for natural produced antibodies, thus immunization against these antigens can produce antibodies that react with the natural viral antigens.

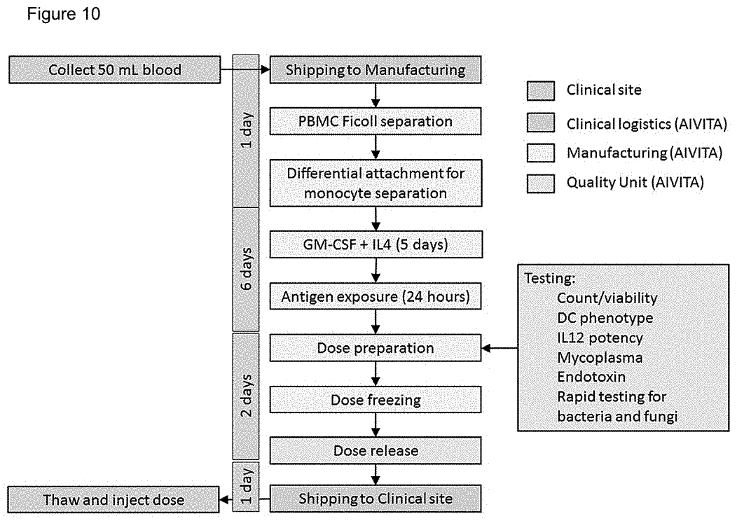

The experiments also demonstrate the lack of direct toxicity of the antigens on the dendritic cells and the activation of the lymphocytes in a cytotoxic manner. The clinical manufacturing (FIG. 10) was validated from additional six patient samples. The manufacturing used all the materials and reagents pre-packaged in individual patient-specific kits. The logistics to maintain the chain of custody of the blood collection and final doses were incorporated in the labeling system of the components.

For practicality, the individual kits were split in four parts, part A and B being used at the site manufacturing according to storage conditions, while part C and D to be used at the clinical site for blood collection and dose administration.

Each kit includes GMP compliant documentation and is anticipated that the collected data is centralized in company's databases for traceability. The present disclosure also contemplates site training materials

The electronic GMP documentation and data collection software is installed at each site and has central reporting capabilities. The software has the capability to be updated remotely, thus avoiding implementation of the local document change control system.

The assembled kits can be reserved ahead of a vaccination season by the manufacturing site(s), in a business model to ensure coverage of materials and avoidance of supply chain depletion during peak demand.

### Example 2. Safety and Efficacy of Autologous DC Vaccine Loaded with Recombinant Full SARS-CoV-2 Spike Protein

A clinical trial using an autologous vaccine product was made from 40 mL peripheral blood lymphocytes subjected to DC differentiation from the contained monocytes and loaded with 0.1, 0.33 or 1 μg SARS-CoV-2 full length recombinant spike protein was conducted. A broad inclusion criteria was used for the trial, excluding only patients with unstable medical conditions and some protected categories of individuals (i.e, children, pregnant women, physically, socially and mentally incapacitated individuals). The primary outcomes evaluated were safety by clinical and laboratory evaluation and efficacy by surrogate marker.

A vector encoding the SARS-CoV-2 spike protein may include a signal sequence and may include a His-tag or other sequence to facilitate purification, but these portions are typically absent from the mature recombinant protein. In some embodiments, the mature full length recombinant spike protein of SARS-CoV-2 has the amino acid sequence:

(SEQ ID NO: 1)
QCVNLTTRTQLPPAYTNSETRGVYYPDKVFRSSVLHSTQDLFLPEFSNV
TWFHAIHVSGTNGTKREDNPVLPFNDGVYFASTEKSNIIRGWIFGTTLD
SKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTP
INLVRDLPQGFSALEPLVDLPIGINITRFQTLLALHRSYLTPGDSSSGW
TAGAAAYYVGYLQPRTFLLKYNENGTITDAVDCALDPLSETKCTLKSFT
VEKGIYQTSNFRVQPTESIVRFPNITNLCPFGEVFNATRFASVYAWNRK
RISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSL
GAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAA
RDLICAQKFNGLTVLPPLLTDEMIAQYTSALLAGTITSGWTFGAGAALQ
IPFAMQMAYRFNGIGVTQNVLYENQKLIANQFNSAIGKIQDSLSSTASA
LGKLQDVVNQNAQALNTLVKQLSSNFGAISSVLNDILSRLDPPEAEVQI
DRLITGRLQSLQTYVTQQLIRAAEIRASANLAATKMSECVLGQSKRVDF
CGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEV
AKNLNESLIDLQELGKYEQYIKWPGYIPEAPRDGQAYVRKDGEWVLLST
FLLEVLFQGP

Manufacturing followed the methods described herein. For each subject a unique identified kit was assigned, the same identification receipt was given to the subject. The PBMCs were separated using a 1.073 density gradient Ficoll reagent.

The PBMCs were differentiated for 5 days in PRIME-XV Dendritic Cell Maturation chemically-defined medium (FujiFilm Irvine Scientific) or AIM-V media (Thermo Fisher) in the presence of GM-CSF at 250 μg/L and IL4 at 100 μg/L, in a 25 mL in a VueLife bioprocess bag (Saint Gobain). On day 5, antigen at 0.1, 0.33 or 1 μg total amount was introduced in the bag. After 2 days, the cells were harvested and doses prepared by re-suspending the cells in autologous plasma. To harvest, the cells were sedimented by centrifugation, the supernatant aspirated, the cells washed by resuspension in saline followed by centrifugation and aspiration of the supernatant. The wash removed media components and any free antigen. Doses were stored at 4° C. and administered the next day by subcutaneous injection.

The subjects were observed after injection for 3 hours, then daily for next 3 days and weekly for the next 4 weeks. Blood was collected for safety labs and for surrogate efficacy.

Surrogate efficacy was tested by ELISPOT for interferon gamma in non-stimulated vs antigen stimulated condition. This assay detects antigen-specific activated IFN-gamma secreting cells and the assay was performed by collecting 8 mL blood in a CPT Vacutainer (Becton Dickinson), PBMC separated by centrifugation and plated in standardized concentration in the presence or absence of spike protein antigen and IL-2 in 24 well plates. After 10 days with no further antigen stimulation, the cells were transferred in predetermined concentration in 96-well ELISPOT plates, each condition in triplicate wells. The spots were stained and counted according to manufacturer's standard procedure (Becton Dickinson).

A total of 138 subjects received doses; 216 were screened. There were 61 subjects who had at least one adverse event (AE). There were a total of 100 adverse events. All events were considered mild (94%) to moderate (6%).

There were no severe or serious adverse events recorded. No subjects discontinued due to an adverse event.

None of the subjects contracted symptomatic COVID-19 disease post-vaccination.

The most common AE were localized injection site reactions as common to other vaccines (Table 2).

TABLE 2
Adverse Events

| | |
|---|---|
| Total Number of AEs | 100 |

Toxicity

| | |
|---|---|
| Grade 1 (mild) | 94 |
| Grade 2 (moderate) | 6 |
| Grade 3 | 0 |
| Grade 4 | 0 |
| Number of SAEs | 0 |
| Discontinued Study | 0 |

Description of AE

| | | |
|---|---|---|
| Injection site reactions | 23 | 23.0% |
| Drowsiness | 15 | 15.0% |
| Flu like symptoms | 9 | 9.0% |
| Headache | 6 | 6.0% |
| All others | <5 | 47.0% |

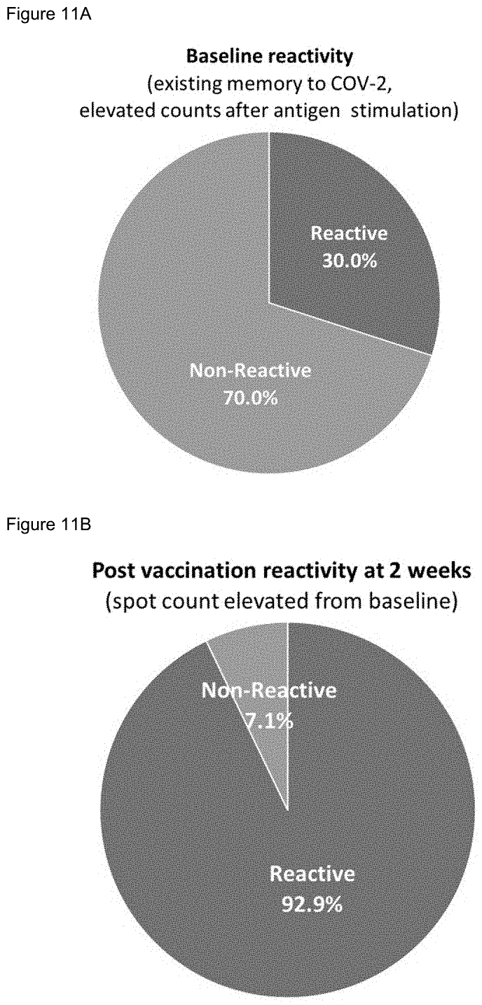

The baseline ELISPOT data demonstrates that 30% of subjects have a history of SARS-CoV-2 exposure (natural infection, or undisclosed vaccine), however none of the subjects presented antibodies at screening (FIG. 11A). As recombinant SARS-CoV-2 spike protein was used as the stimulant, positive reactions are expected to reflect actual exposure to SARS-CoV-2 and not cross-reactivity with another coronavirus to which the subject may have been exposed.

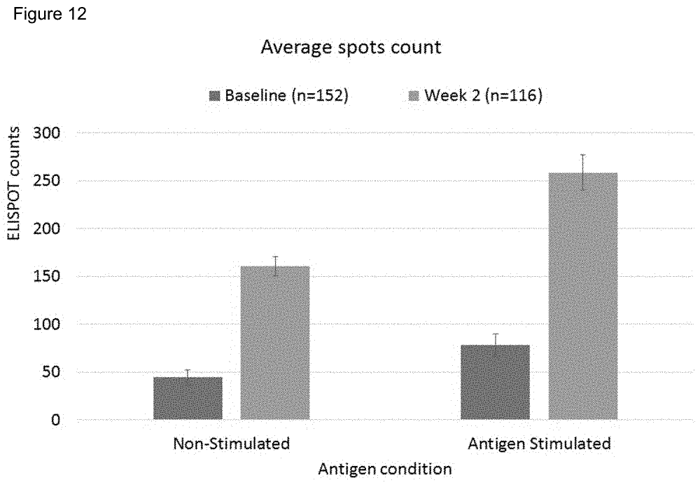

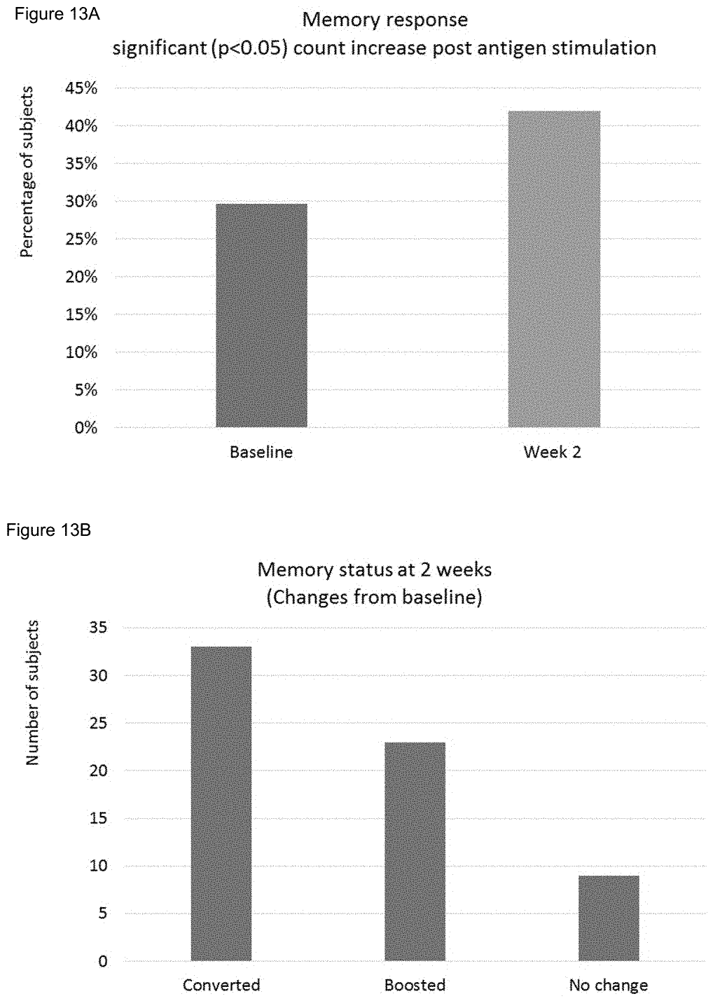

By two weeks post vaccination, the ELISPOT reactivity increased to 92.9% of the tested subjects (FIG. 11B). The average spot count significantly increased (p<<0.001) in both stimulated and non-stimulated conditions (FIG. 12, Table 3), with 43% of the subjects demonstrating SARS-CoV-2 spike protein-specific cytotoxic memory cells (IFNγ secretory lymphocytes; FIG. 13A). The majority of the subjects with cytotoxic memory cells at two weeks post-vaccination are the result of conversion. Some of the subjects with pre-existing reactivity demonstrated a booster effect post-vaccination, while a few remained unchanged (FIG. 13B).

TABLE 3
Average Spot Counts

| | |
|---|---|
| Non-Stimulated | Antigen Stimulated |

| Time point | Count | SEM | Count | SEM |
|---|---|---|---|---|
| Baseline (n = 152) | 44 | 8 | 78 | 12 |
| Week 2 (n = 116) | 161 | 10 | 258 | 18 |

The ELISPOT data demonstrate a high reactivity post-vaccination at two weeks suggestive of a primary immune response in a majority of the subjects.

Data from a 28 patient pilot safety study showed that the cytotoxic memory response persists at least four months post vaccination, however it is expected to last much longer.

In conclusion these data are indicative of induction of a cell mediated immunity.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A personalized vaccine kit comprising,
a. a kit container,
b. blood collection supplies,
c. monocyte separation media or an inertial microfluidic device,
d. dendritic cell (DC) differentiation media components,
e. a cell culture container with at least one access port,
f. indicia of unique identity, and
g. an antigen.

2. The personalized vaccine kit of claim 1, wherein the kit container comprises insulated walls and is capable of serving as an incubator.

3. The personalized vaccine kit of claim 2, wherein the incubator comprises a power supply and a thermostat system.

4. The personalized vaccine kit of claim 3, wherein the thermostat system comprises a phase exchange material and a positive temperature coefficient (PTC) material, but does not comprise a temperature controller.

5. The personalized vaccine kit of claim 1, wherein the DC differentiation media components are provided dry in the cell culture container.

6. The personalized vaccine kit of claim 1, wherein the DC differentiation media components comprise IL-4.

7. The personalized vaccine kit of claim 6, wherein the DC differentiation media components comprise GM-CSF.

8. The personalized vaccine kit of claim 1, wherein the DC differentiation media components comprise IFNγ, IFNα, IL-2, or any combination thereof.

9. The personalized vaccine kit of claim 1, wherein the antigen is SARS-CoV-2 full-length recombinant spike protein.

10. A method of making a personalized, autologous dendritic cell (DC) vaccine using the kit of claim 1, comprising:
a) collecting from 5 to 50 mL of blood from an individual,
b) isolating peripheral blood mononuclear cells (PBMC) from the blood,
c) differentiating the PBMC to generate immature DC by adding the DC to the cell culture container and incubating the cells for 2-5 days, then
d) adding antigen to the cell culture container to load the immature DC with antigen and incubating for a further 1-2 days, and
e) harvesting the antigen-loaded immature DC.

11. The method of claim 10, further comprising reserving autologous plasma from the isolating step.

12. The method of claim 10, further comprising re-suspending the harvested antigen-loaded immature DC in autologous plasma.

13. The method of claim 10, further comprising affixing the indicia of unique identity to a container containing cells or plasma from the individual.

14. The method of claim 10, further comprising storing the harvested, re-suspended antigen-loaded immature DC prior to administration to the individual.

15. The method of claim 14, comprising storing the harvested, re-suspended antigen-loaded immature DC at room temperature for up to 6 hours.

16. The method of claim 14, comprising storing the harvested, re-suspended antigen-loaded immature DC at 4° C. for up to 48 hours.

17. The method of claim 10, further comprising re-suspending the harvested antigen-loaded immature DC in a mixture autologous plasma and cryopreservative and storing the harvested, re-suspended antigen-loaded immature DC at −80° C. for up to 21 days.

18. A personalized, autologous dendritic cell (DC) vaccine made by the method of claim 10.

19. A method of immunizing an individual comprising administering the personalized, autologous dendritic cell (DC) vaccine to the individual by subcutaneous or intradermal injection.
